SEQUENCE LISTING (17 sequences)

SEQ ID NO: 1 - DNA, 1170 bp, Artificial sequence
  synthetic fdh gene
atggcaaaagtactttgcgttctttatgatgatccggtagatggctatccgaagacgtatgcccgagatgatttaccgaagatagatcactatccaggagggcaaacattgccgacgccgaaagcaattgacttcacgcctgggcaattgttaggaagcgtatctggcgagctgggacttagaaagtatcttgagtccaatggacatacgttagtggtaactagcgataaggatgggccagactcagtgtttgaacgggagttagtggatgccgatgttgtcattagtcaaccgttctggcctgcatatcttacgccggaaagaatcgcaaaagcgaagaacttgaaactagccctgacagcaggaattggaagcgatcatgtggatttgcaaagcgctattgatcgcaatgttaccgtggcagaggtgacatattgtaattctattagtgtagctgagcatgtggtaatgatgattttatccttagttagaaattacttgccgagccacgaatgggctcgtaaaggcgggtggaatattgcagattgcgtttctcatgcttatgatttagaggcgatgcatgttggcacggttgcggcgggacgtataggcttggcagtcttgcgtcgactagcaccgtttgacgttcatttacactatactgatcgacatcgtcttccagagtccgttgagaaagaacttaacttaacctggcatgcaacgcgtgaagatatgtatccggtgtgtgacgtggtaacattgaattgcccgttacatcctgaaactgaacacatgatcaatgacgaaacgttgaaactgtttaaacgaggcgcttatatcgtaaacacagccagagggaaactttgtgatcgggatgctgtagccagagcacttgagagcggacgcttagccgggtatgcaggcgacgtgtggtttccacaacctgccccgaaagatcatccgtggagaacgatgccgtataatggaatgacgccacatatttcaggcactacgttaacagcacaagcacgttatgcggccggcacccgtgaaattcttgagtgcttcttcgaaggccgtccgatccgagatgaatatttgattgtacaaggtggcgcattagctgggacaggagcacatagttatagcaaaggcaatgctacgggaggc

SEQ ID NO: 2 - DNA, 1558 bp, artificial sequence
  synthetic fdh gene including promoter and terminator
gcctctagaagggcaatctgaaaggaagggaaaattcctttcggattgtccttttagttatttttatggggagtgaatattatataggcattacggaaatgataatggcagagttttttcatttattagactgcttgatgtaattggatgtgatgatacaaaaataatgttgtgtaaacaaaatgttaacaaaaaagacaaatttcattcatagttgatacttgataaagattgtgaaataatgcacaatatatcaatgtatgagcagtttcacaaattcattttttggaaaggatgacagacagcgatggcaaaagtactttgcgttctttatgatgatccggtagatggctatccgaagacgtatgcccgagatgatttaccgaagatagatcactatccaggagggcaaacattgccgacgccgaaagcaattgacttcacgcctgggcaattgttaggaagcgtatctggcgagctgggacttagaaagtatcttgagtccaatggacatacgttagtggtaactagcgataaggatgggccagactcagtgtttgaacgggagttagtggatgccgatgttgtcattagtcaaccgttctggcctgcatatcttacgccggaaagaatcgcaaaagcgaagaacttgaaactagccctgacagcaggaattggaagcgatcatgtggatttgcaaagcgctattgatcgcaatgttaccgtggcagaggtgacatattgtaattctattagtgtagctgagcatgtggtaatgatgattttatccttagttagaaattacttgccgagccacgaatgggctcgtaaaggcgggtggaatattgcagattgcgtttctcatgcttatgatttagaggcgatgcatgttggcacggttgcggcgggacgtataggcttggcagtcttgcgtcgactagcaccgtttgacgttcatttacactatactgatcgacatcgtcttccagagtccgttgagaaagaacttaacttaacctggcatgcaacgcgtgaagatatgtatccggtgtgtgacgtggtaacattgaattgcccgttacatcctgaaactgaacacatgatcaatgacgaaacgttgaaactgtttaaacgaggcgcttatatcgtaaacacagccagagggaaactttgtgatcgggatgctgtagccagagcacttgagagcggacgcttagccgggtatgcaggcgacgtgtggtttccacaacctgccccgaaagatcatccgtggagaacgatgccgtataatggaatgacgccacatatttcaggcactacgttaacagcacaagcacgttatgcggccggcacccgtgaaattcttgagtgcttcttcgaaggccgtccgatccgagatgaatatttgattgtacaaggtggcgcattagctgggacaggagcacatagttatagcaaaggcaatgctacgggaggcagcgaggaagcagctaaatttaagaaagcggtttaacacagcaggggctgatcggcccctgttatgtttcattctagagcc

SEQ ID NO: 3 - protein, 401 aa, Pseudomonas
MAKVLCVLYDDPVDGYPKTYARDDLPKIDHYPGGQTLPTPKAIDFTPGQLLGSVSGELGLRKYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTDRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAV

SEQ ID NO: 4 - DNA, 307 bp, Bacillus stearothermophilus
gcctctagaagggcaatctgaaaggaagggaaaattcctttcggattgtccttttagttatttttatggggagtgaatattatataggcattacggaaatgataatggcagagttttttcatttattagactgcttgatgtaattggatgtgatgatacaaaaataatgttgtgtaaacaaaatgttaacaaaaaagacaaatttcattcatagttgatacttgataaagattgtgaaataatgcacaatatatcaatgtatgagcagtttcacaaattcattttttggaaaggatgacagacagcg

SEQ ID NO: 5 - DNA, 3006 bp, Bacillus stearothermophilus
tgtaaattatcactttatttccgcacaaaaaagactcttttttgcacattccttcggaatatccctctccccctttccgaaagaatgtgctaaattttttgtgaattatttcggaatgggacatgggtgatttccagaggggggacgaggacgtgattcggtttcatgactttcaggtcgatgtccagacatatgcccagcggggaaaacaaaacgactttccccttcttaagcggtgccctcattgccaggcaaaacgccctctttatcgccatgggtactacgaacgaaatgccgtgacgtcgcatcagtcttatcgcatttggatcgctcggtatcgctgcccggagtgcaggaggacggtggccgtgttgccttcatttcttctcccttattttcagtatacgttgcccaccatatggagagtggtgaaagaacggttgggcctgactccgaaacgggggatggaggaggctccactccttcctacggatgaaggggttttattttatgtcccgacgtttattccgaaatttgaaccaccttcattggttttttgcggagcgctggagaaaaattggtcctgccatcgcccaagccgagagaacgagccctatggtggatccagacgatggaggagatcggcctctttttcgtcatccaagagatatgggagcaccgatcgacgcatctttttgcacgtacattcagttcctgatttacttatatccccttatatggaatcatttatagattcccaaacctttcctctcgacggtcgggggaatgatccgataggatagagacaggatggaccgataaggtcctagaatgggatgaacgaaggaggagatcgaaatgaatgagtcgatgagacaggagatcgctttatttcggtatggattgatcgctccattggtgaatggacaagtcgatccaaaacgtacttgaaggaagtagcggaacggatccatcaagttccccaccatggagagaaacgcatcgccgccaaaacgatcctcgactggtgcacgcagtacaaaaaagggggctttgaggcgctgaagccgaaacgacggtcggaccgtggccattcccggaggctgtcacctgaagaagaggatcacattttagccctgagaaaaaaacacccccacatgcccgtgacggtgttttaccaacaccttatcgagcagggggaaatccaatccatctcttatttcactatataccgacttttaaaaaaatacaacctcgtggggaaagaaattttaccgattcctgaacgaaaacgattcgcgtacgatcaggtcaatgagctctggcaaggtgatttgtcccatggcccgttgattcgcgtgaatggcaaaacgcaaaaaacgtttttgattgcctatatcgatgactgctcgcgggtcgtgccgtacgctcagtttttctcttccgagaaatttgacgggttgcggatcgtaaccgcgcggagttagggatcaccttgatccatacccagccgtacgatccgcaaagcaaagggaaaatcgaacgatttttccgcaccgtacagacgcggttttacccgttgctcgaaatgaattcaccgaagtcgctcgaagagctaaacgagcgattttggaagtggttggaggaagattaccatcgaaaaccgcatgcctcgttgaacgggaagacgccacatgaagtgtttcaatcgcaagtccatttggtgtcgttcgtcgaggattcggattggctcgactcgatctttttgaaacgcgaataccgtaaagtgaaggccgatggtacggtcacgttgaacaagcagctgtatgaagttccgccccggttcatcggacaatcgatcgaactccgttatgatgaacaaggcgtgtatgtgtacgaagacggtcaacgggtcgccgaagcggtccttgttcgcttcgaggacaatgcctatgtgaaacgccatcggtcaccgtttgcggcggttccggtagagggaggcgaaaacgatgtataaaacgttttattccctttcccgagagccgttttcgaaggagacgaatccaccagaggcttatcaaggggcctcgtatcaagaggccctcgccgctttggactacgtgaaacgaacaagagggatcgggctattgatcggtgaaccaggggccggcaagacattcgcccttcgggcgtttaaggaatccctgaatccgtcactgtatcacgtcgtttattttccattgtcaacgggaagcgtgatggacttttatcgcggccttgccttcgggctcggggaagagccgaaataccgcaaggtcgacttgttttatcaaatccaacaagggatcgagcgcttgtatcatgaacaacgggtaacgtcagtgttcatcctcgatgaaatgcatttagcgaaggatgcctttctgcaggatatcgcgatcctgttcaactttcacatggactcaacaaatccgtttgtcttgattttggcggggctgccccatttacaggcaaaactacggttgaatcaacaccgtccgcttcaccaacgaatcatcatgcgataccagatggggcctcttgataaggaagaagtggtaggatatatcgaacaccgcctgaaacaggcgggggcgaaacacccgatttttaccccagctgccttagaagcgatcgccctgcagtcgcaggggtggccgcggatcatcaacaacctcgccaccacttgcctgttatacggcgctcaattaaaaaaacatatgattgacgaagacattgtgcgtatggcagccgaagaaatggggtactgacacagcaggggctgatcggcccctgttatgtttcatcccgatccatcctcattctagttaatcatccgaaataatgtgcaaatgttcggaaataatctgcaaaacctggaataattcgcaaagattttgcacattatttccgaatccgtccgaaataatttgaaaaagggattctgaaataatgtgctaatttaca

SEQ ID NO: 6 - DNA, 26 bp, Artificial sequence
  primer
agtactgaaatccggatttgatggcg

SEQ ID NO: 7 - DNA, 24 bp, Artificial sequence
  primer
agtactgctaaatttccaagtagc

SEQ ID NO: 8 - DNA, 29 bp, Artificial sequence
  primer
gaacgattctagatacagcaagattccgc

SEQ ID NO: 9 - DNA, 26 bp, Artificial sequence
  primer
gtttgcgaattcatagacggacgcag

SEQ ID NO: 10 - DNA, 28 bp, Artificial sequence
  primer
acacagacgtcggcgatttgattcatac

SEQ ID NO: 11 - DNA, 34 bp, Artificial sequence
  primer
cgccatgacgtccatgataattactaatactagg

SEQ ID NO: 12 - DNA, 29 bp, Artificial sequence
  primer
agggcaatctgaaaggaagggaaaattcc

SEQ ID NO: 13 - DNA, 27 bp, Artificial sequence
  primer
tgcacagatctccaccaaatcggcgtc

SEQ ID NO: 14 - DNA, 29 bp, Artificial sequence
  primer
ttgagcagatcttgatgcaaaacgataac

SEQ ID NO: 15 - DNA, 25 bp, Artificial sequence
  primer
taaagccgatgagcagcagttgaag

SEQ ID NO: 16 - DNA, 33 bp, Artificial sequence
  primer
atattatctagacattacggaaatgataatggc

SEQ ID NO: 17 - DNA, 27 bp, Artificial sequence
  primer
tcacaatctagacaatcggccataaac

(19) United States
(12) Patent Application Publication
(10) Pub. No.: US 2009/0226992 A1
(43) Pub. Date: Sep. 10, 2009

(54) Enhancement of Microbial Ethanol Production

(75) Inventors: Muhammad Javed, Essex (GB); Namdar Baghaei-Yazdi, London (GB)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Bioconversion Technologies Limited, Surrey (GB)

(21) Appl. No.: 12/294,084

(22) PCT Filed: Mar. 26, 2007
(86) PCT No.: PCT/GB07/01060

§ 371 Date: Apr. 17, 2009

(30) Foreign Application Priority Data
Mar. 24, 2006 (GB) 0605890.3

(51) Int. Cl. C12P 7/06; C12N 1/00; C12N 1/20; C07H 21/00

(52) U.S. Cl. 435/161; 435/243; 435/252.5; 536/23.2

(57) ABSTRACT

A thermophilic microorganism lacks lactate dehydrogenase activity and preferably contains an active pyruvate formate lyase pathway. The thermophilic microorganism contains a gene encoding an NAD-linked formate dehydrogenase. The gene encoding an NAD-linked formate dehydrogenase is preferably a codon optimised version of the gene encoding a thermostable NAD-linked formate dehydrogenase. DNA constructs allow stable expression of the gene encoding an NAD-linked formate dehydrogenase in the thermophilic microorganism. The DNA constructs are based upon use of an insertion sequence to achieve stable expression or recombination to insert the gene encoding an NAD-linked formate dehydrogenase into the lactate dehydrogenase gene, thus achieving gene knockout and new functionality in a single step. The microorganisms are useful in fermentation of sugars to produce ethanol.

### FIELD OF THE INVENTION

This invention relates to fermentation procedures and microorganisms for use therein and in particular to the enhancement of microbial ethanol production. More specifically, the invention relates to enhanced ethanol production by thermophilic bacteria, such as Bacilli from mixed sugars derived from the hydrolysis of biomass. In particular, the invention envisages a novel pathway for ethanol production by cloning a gene which encodes an NAD-linked formate dehydrogenase enzyme into a microorganism that possesses a functional gene which encodes a pyruvate-formate lyase enzyme complex but lacks lactate dehydrogenase activity.

### BACKGROUND TO THE INVENTION

Bioethanol is currently made from glucose, maltose or sucrose derived from cereal starch, sugar cane or sugar beet, which all have food value. Celluloses and hemicelluloses form a major part of agricultural by-products and could, in principle, be a major source of low-cost, renewable bio-ethanol. However, it is difficult and expensive to derive fermentable sugars from cellulose. In contrast, hemicelluloses are almost as abundant as cellulose and are easy to hydrolyse, but yield a mixture of mainly pentose sugars that yeasts cannot ferment.

For this reason, Hartley (see International Publication Number WO 88/09379) proposed production of ethanol by mutants of a thermophilic Bacillus, which very rapidly ferments all of the sugars derived from biomass, at temperatures up to 70° C. High ethanol yield is achieved only by stressed and moribund cells, however.

Many micro-organisms contain a pyruvate-formate lyase (PFL) pathway that converts pyruvate into acetyl CoA and formate (FIG. 1A). Heterolactate fermentative microorganisms are one such class. These microorganisms first convert input sugars to pyruvate (generally by the EMP pathway of glycolysis), which then can take many routes to produce lactate, formate, acetate, ethanol and CO₂, in various proportions, depending on the growth conditions.

In fully aerobic cells, the pyruvate is normally metabolised to H₂O and CO₂ via the pyruvate dehydrogenase (PDH) pathway, tri-carboxylic acid cycle and the Electron Transport Chain. However, in many of these organisms, particularly thermophilic Bacilli, sugar uptake and glycolysis appear to be unregulated and lactate is a dominant product at high sugar concentrations, even under aerobic conditions. This suggests that the PDH flux has then become saturated, and that the excess pyruvate is diverted into an overflow lactate dehydrogenase pathway. This is not used for growth but produces heat which causes the ambient temperature to rise and kills mesophilic competitors, as can be seen when fresh grass is put on a compost heap.

If the ldh gene (encoding lactate dehydrogenase) is inactivated, as described for example in WO 02/29030, lactate production stops and the excess pyruvate is diverted mainly into the growth-linked PFL pathway, (FIG. 1A). However, at very high sugar concentrations and/or at acid pH, the PFL pathway flux declines and the excess pyruvate then overflows into an anaerobic PDH pathway, which yields only ethanol and CO₂ (FIG. 1B). Therefore the preferred conditions to obtain high ethanol yields are those that reduce flux through the PFL pathway and increase flux via the PDH pathway (Hartley, B. S, and Shama, G. Proc. Roy. Soc. Lond. 321, 555-568 (1987)). Unfortunately, under such conditions the cells experience metabolic stress, with reduced ATP production, and a potential imbalance in NAD/NADH and CoA/acetyl CoA ratios (FIG. 1C).

Various fermentation protocols have been proposed to try to avoid or minimize this problem such as that of Hartley, B. S. as discussed above (see International Publication Number WO 88/09379).

There are two classes of formate dehydrogenase. One (encoded by the fdhF gene) converts formate into CO₂+H₂ and is typical of enterobacteriae such as E. coli. The other (encoded by the fdh1 gene) converts formate+NAD into CO₂+NADH₂ and is present in many facultative anaerobes. Berrios-Rivera et al (Metabolic Engineering 4, 217-219 (2002) replaced the fdhF gene in E. coli with a yeast fdh1 gene and found that the reduced anaerobic products such as ethanol, lactate and succinate increased relative to oxidised products such as acetate. Building on this observation, San, K-Y. Berrios-Rivers, S. J. and Bennett, G. N. (see International Publication Number WO 2003/040690 proposed the introduction of an NAD-linked formate dehydrogenase gene as a general method to increase reducing power in cells involved in a broad range of bio-transformations. Subsequently San, K-Y. Bennett, G. N. and Sanchez, A. (US Patent Application US 2005/0042736 A1) proposed a specific application of this concept for production of succinate. These studies were carried out in E. Coli, an example of a mesophile where sugar uptake is regulated. The purpose of these experiments was to increase intracellular NADH levels so as to provide enhanced reducing power for various bio-transformations.

The yeast formate dehydrogenase recommended by Sen. et al (2004) is inactive at 60° C., which is the minimal growth temperature for the thermophilic bacteria potentially of use in bioethanol production. The most thermostable formate dehydrogenases so far described is the Pseudomonas sp. 101 enzyme (A. Rojkova, A. Galkin, L. Kulakova, A. Serov, P. Savitsky, V. Fedorchuk, V. Tishkov FEBS Letters, Volume 445, Issue 1, Pages 183-188, 1999).

### SUMMARY OF THE INVENTION

The present invention attempts to solve the problems of producing high yields of ethanol from biomass. In particular, herein described for the first time is a novel metabolic pathway which allows thermophilic microorganisms, especially bacteria such as Bacillus to produce maximal ethanol yields.

The invention relies upon microorganisms which lack lactate dehydrogenase activity and thus require an alternative route for re-oxidation of excess NADH produced by glycolysis. This is provided by introduction into the microorganism of a gene encoding an NAD-linked formate dehydrogenase, such as an fdh1 gene. In thermophilic microroganisms, and in contrast to mesophiles such as E. coli, sugar uptake is unregulated and this leads to accumulation of NADH in the presence of high levels of sugars. This eventually leads to a metabolic collapse and so-called “redox death” as shown schematically in FIG. 1C. Incorporation into the microorganism of a gene encoding an NAD-linked formate dehydrogenase helps to prevent cell death at high sugar concentrations by leading to a decrease in NADH levels and an increase in NAD levels. This is partly by restoring flux through the pyruvate dehydrogenase (PDH) pathway but most importantly, inclusion of a gene encoding an NAD-linked formate dehydrogenase creates a novel pyruvate formate lyase (PFL)-NAD-linked formate dehydrogenase (FDH) pathway for ethanol production. FIG. 1D shows the potential for this PFL-FDH pathway to restore redox balance by converting all of the pyruvate produced by rapid glycolysis in the presence of high sugar levels to ethanol and CO₂, especially under neutral pH conditions. Importantly, the pathway operates under conditions that are optimal for cell growth, leading to rapid ethanol production and high yield, since the PFL pathway is the major growth-linked anaerobic pathway in thermophilic microorganisms.

Accordingly, in a first aspect the invention provides a microorganism, in particular a thermophilic microorganism, lacking lactate dehydrogenase (ldh) activity, characterised in that the microorganism, preferably a thermophilic microorganism contains a gene encoding an NAD-linked formate dehydrogenase (fdh).

In one embodiment, the microorganism lacks lactate dehydrogenase activity by virtue of an appropriate gene deletion or other mutation which removes lactate dehydrogenase activity. Thus, preferably the ldh gene is deleted or otherwise rendered non-functional. Methods of gene knock-out and deletion are well known in the art and preferred examples are described in detail herein. Moreover, known strains of bacteria lacking lactate dehydrogenase activity (such as TN-T9 deposited under accession number NCIMB 41075 and TN-TK deposited under accession number NCIMB 41115) may be suitable for use in the present invention.

The microorganism of the invention typically contains an active pyruvate formate lyase pathway. In particular, the microorganism preferably comprises a gene encoding a pyruvate formate lyase such as the pf1 gene. The microorganisms of the invention typically also contain an active pyruvate dehydrogenase (PDH) pathway.

In a preferred embodiment, the gene encoding an NAD-linked formate dehydrogenase is integrated into the genome of the thermophilic microorganism. However, it is also possible for stable expression to be achieved without integration for example by introduction of a suitable plasmid. One preferred method of integration is by recombination. The gene encoding an NAD-linked formate dehydrogenase may be operably linked to any suitable regulatory element to direct expression of the NAD-linked formate dehydrogenase. By “operably linked” is meant a functional linkage exists between the regulatory element and the gene encoding an NAD linked formate dehydrogenase. For example, the gene encoding an NAD-linked formate dehydrogenase may be linked to a suitable promoter which may be a constitutive or inducible promoter for example. “Promoter” is defined herein to include a region of DNA which is involved in the binding of RNA polymerase to initiate transcription.

Typically the promoter is a prokaryotic promoter and thus includes the appropriate −10 and −35 sequences, the consensus sequences of which are well defined in the art. The gene may also be operably linked to other appropriate regulatory sequences such as terminators for example. “Terminator” is defined as a nucleotide sequence which causes RNA polymerase to terminate transcription. In one embodiment, the gene encoding an NAD-linked formate dehydrogenase is expressed from its own promoter. In an alternative embodiment, the gene encoding an NAD-linked formate dehydrogenase is expressed from a promoter of the thermophilic microorganism (due to integration in an appropriate location in the genome). Constructions can also be envisaged where expression of the gene encoding an NAD-linked formate dehydrogenase is driven by a foreign promoter. This may be done to achieve maximal expression levels or inducible expression for example. As an example, phage promoters such as T7 may be utilised in conjunction with a suitable phage polymerase (which may be provided in a separate or the same DNA construct).

In a particularly preferred embodiment, the gene encoding an NAD-linked formate dehydrogenase is operably linked to the appropriate regulatory regions of a gene encoding a lactate dehydrogenase, in particular the upstream regulatory regions. The regulatory region preferably comprises the promoter of a gene encoding a lactate dehydrogenase. The promoter may be defined to include as a minimum functional unit the appropriate −10 and −35 sequences. Thus, according to one preferred embodiment of the invention the gene encoding an NAD-linked formate dehydrogenase is inserted into the lactate dehydrogenase gene of the thermophilic microorganism, thus inactivating the lactate dehydrogenase activity of the thermophilic microorganism. This embodiment is particularly preferred since both modifications required to produce a thermophilic microorganism of the invention are produced in the same step. Suitable constructs for achieving this are described in detail herein.

Ethanol production by thermophilic bacteria is advantageous since it can be carried out at high temperatures. Whilst thermophilic microorganisms have lower ethanol tolerance than yeasts, ethanol may be continuously and conveniently removed from the high temperature fermentation by membrane and/or mild vacuum evaporation. In optimal anaerobic growth conditions, Bacillus strain LLD-R grows very rapidly at 70° C. almost exclusively by the PFL-pathway (Hartley and Shama, 1982). It can be envisaged that growth by the novel PFL-FDH pathway would be equally vigorous, but the maximum growth temperature would be limited by the thermostability of the NAD-linked formate dehydrogenase introduced into the thermophilic microorganism. Accordingly, in one preferred embodiment, the thermophilic microorganism of the invention incorporates a gene encoding a thermostable NAD-linked formate dehydrogenase and/or a gene whose nucleotide sequence has been codon optimised to facilitate expression by a thermophilic microorganism. Production of such a thermostable NAD-linked formate dehydrogenase is described in detail herein. In a specific embodiment, the gene encoding an NAD-linked formate dehydrogenase comprises, consists essentially of or consists of the nucleotide sequence set forth as SEQ ID NO: 1. In a further embodiment, the thermophilic microorganism of the invention incorporates a codon optimised for expression in Bacillus, gene encoding a thermostable NAD-linked formate dehydrogenase comprising, consisting essentially of or consisting of the nucleotide sequence set forth as SEQ ID NO:2. This sequence includes, in addition to the basic thermostable NAD-linked dehydrogenase sequence, promoter and terminator regions and also Xba1 sites to facilitate cloning of the gene into a suitable DNA construct.

In a still further embodiment the gene encoding an NAD-linked formate dehydrogenase is the fdh1 gene. The fdh1 gene may be derived from any suitable source and is preferably codon optimised for expression in the relevant thermophilic microorganism.

The thermophilic microorganism of the invention may be produced by transformation with any of the DNA constructs of the invention as described in further detail herein. Accordingly, the discussion provided there applies mutatis mutandis to this embodiment of the invention.

The thermophilic microorganism of the invention may be any suitable microorganism for production of ethanol from biomass. Preferably, the thermophilic microorganism is a heterolactate fermentative microorganism. More preferably the thermophilic microorganism is a thermophilic bacterium and is more preferably of the genus Bacillus and even more preferably Bacillus stearothermophilus. In one embodiment, the thermophilic microorganism of the invention is derived from the known strain LLD-R or LLD-15 (of Bacillus stearothermophilus). In a further embodiment, the thermophilic microorganism is Geobacillus thermoglucosidasius.

The fermentation processes facilitated by the present invention preferably utilise a synthetic NAD-linked formate dehydrogenase, designed to express a thermostable amino acid sequence due to use of the codon preferences of the appropriate thermophilic microorganism such as Bacillus strain LLD-R. The synthetic gene preferably contains engineered restriction sites to assist insertion into the lactate dehydrogenase gene. Thereby deletion of the LDH pathway and creation of the PFL-FDH pathway are achieved in a single operation. Accordingly, in a second aspect, the invention provides a thermostable NAD-linked formate dehydrogenase. Preferably, the thermostable NAD-linked formate dehydrogenase remains functional at or above a temperature of 60° C. Preferably, the thermostable enzyme is encoded by a nucleotide sequence which has been codon optimised for expression in a thermophilic microorganism. The formate dehydrogenase may comprise, consist essentially of or consist of the amino acid sequence set forth as SEQ ID NO: 3 in one embodiment.

A specific thermostable NAD-linked formate dehydrogenase has been designed based upon the amino acid sequence of the Pseudomonas sp 101 formate dehydrogenase (SEQ ID NO:3) and through use of optimised codons for Geobacillus thermoglucosidasius as discussed in more detail in the detailed description below. The skilled person will appreciate that derivatives of this basic sequence will retain functionality. For example, conservative and semi-conservative substitutions may result in thermostable NAD-linked formate dehydrogenases and these derivatives are intended to fall within the scope of the invention provided they retain effective catalytic activity and thermostability such that they are useful in ethanol production using thermophilic microorganisms. Similarly, minor deletions and/or additions of amino acids may produce derivatives retaining appropriate functionality.

In a third aspect, the invention relates to a synthetic gene encoding a thermostable NAD-linked formate dehydrogenase. Preferably the gene comprises, consists essentially of or consists of the nucleotide sequence set forth as SEQ ID NO:1. This sequence represents a novel fdh gene sequence in which the codons are optimised for production of a thermostable NAD-linked formate dehydrogenase. In a more specific embodiment, the gene encoding a thermostable NAD-linked formate dehydrogenase comprises, consists essentially of or consists of the nucleotide sequence set forth as SEQ ID NO:2. This sequence incorporates the coding region for the thermostable NAD-linked formate dehydrogenase together with a suitable Bacillus promoter and rho-independent terminator. The sequence also incorporates suitable restriction sites to assist in cloning, in particular Xba1 sites. The skilled person will readily appreciate that minor modifications to the nucleotide sequence may be made without altering the functionality or thermostability of the resultant enzyme, for example through replacing optimized codons with other codons which are preferred in the translation systems of the appropriate thermophilic microorganism.

The invention also relates to a DNA construct containing a gene encoding an NAD-linked formate dehydrogenase, in particular a thermostable NAD-linked formate dehydrogenase, wherein the gene is flanked by restriction sites to facilitate cloning of the gene into a suitable DNA construct, such as an expression vector or plasmid.

In a related aspect, the invention also provides a DNA construct comprising a regulatory sequence operably linked to a gene encoding a thermostable NAD-linked formate dehydrogenase. This DNA construct thus facilitates transformation of thermophilic microorganisms, in particular those lacking lactate dehydrogenase activity, in order to produce thermophilic microorganisms capable of efficient fermentation giving maximal ethanol yields. As aforementioned, the term “operably linked” as used herein refers to a functional linkage between the regulatory sequence and the gene encoding the NAD-linked formate dehydrogenase, such that the regulatory sequence is able to influence gene expression. For example, a preferred regulatory sequence is a promoter. As aforementioned, the gene encoding an NAD-linked formate dehydrogenase preferably comprises, consists essentially of or consists of the nucleotide sequence set forth as SEQ ID NO:1. A preferred regulatory sequence is a promoter, although the DNA construct may additionally incorporate suitable terminator sequences. In one specific embodiment, the promoter comprises the nucleotide sequence set forth as SEQ ID NO:4. Other promoters, as discussed above, may be utilised for high levels and/or inducible expression.

In a further aspect of the invention there is provided a DNA construct comprising a gene encoding an NAD-linked formate dehydrogenase and an insertion sequence, wherein the insertion sequence facilitates integration of the gene encoding an NAD-linked formate dehydrogenase into the genome of a thermophilic microorganism transformed with the DNA construct. By “insertion sequence” is meant a transposable DNA element which is capable of integration into the genome of the appropriate thermophilic microorganism. Insertion sequences may also be referred to as insertion sequence elements (IE) and may be naturally occurring. In one specific embodiment of the invention, the insertion sequence is derived from Bacillus stearothermophilus strain LLD-R or LLD-15.

In a more specific embodiment, the insertion sequence comprises, consists essentially of or consists of the nucleotide sequence set forth as SEQ ID NO:5 (FIG. 3). The preferred insertion sequence may be generated by amplification using primers comprising, consisting essentially of or consisting of the nucleotide sequence set forth as SEQ ID NO: 6 and 7. In this case, genomic DNA from the known Bacillus stearothermophilus strain LLD-15 may be used as the template. One particularly preferred DNA construct is plasmid pUB-ISF1 (as described in the experimental section below and in FIG. 5).

In a still further aspect, the invention relates to a DNA construct comprising a (fdh) gene encoding an NAD-linked formate dehydrogenase operably linked to appropriate regulatory regions of a gene encoding a lactate dehydrogenase, in particular the upstream regulatory regions. The regulatory regions preferably comprise the promoter of a gene encoding a lactate dehydrogenase (ldh). The promoter may be defined to include as a minimum functional unit the appropriate −10 and −35 sequences to allow effective RNA polymerase binding. The lactate dehydrogenase gene promoter is suitable for driving high levels of expression in thermophilic microorganisms such as Bacilli and also may advantageously be used as part of the cloning strategy to achieve both deletion of lactate dehydrogenase activity and introduction of NAD-linked formate dehydrogenase activity in the same step. The DNA construct may thus also be defined as comprising a gene encoding an NAD-linked formate dehydrogenase operably linked to a nucleic acid molecule which comprises the promoter of a gene encoding a lactate dehydrogenase (ldh).

The DNA construct preferably also contains part of the coding sequence of the host lactate dehydrogenase gene downstream of the gene encoding an NAD-linked formate dehydrogenase. This facilitates gene integration in a microorganism transformed with the DNA construct. By “at least part” is meant a portion of the gene of sufficient length to allow gene integration into the genome of a microorganism containing the lactate dehydrogenase gene by recombination (preferably by double cross-over). The part of the coding sequence preferably incorporates the end of the lactate dehydrogenase gene. In one embodiment, at least 100, 200, 300, 400, 500, 600, 700 or 750 nucleotides of the lactate dehydrogenase gene are incorporated downstream of the gene encoding an NAD-linked formate dehydrogenase. Thus, in one embodiment of the invention, the DNA construct comprises a gene encoding an NAD-linked formate dehydrogenase wherein the gene encoding an NAD-linked formate dehydrogenase is flanked by nucleotide sequence from a gene encoding a lactate dehydrogenase (derived from the thermophilic microorganism of interest). The flanking sequences are of sufficient length to allow integration of the gene encoding an NAD-linked formate dehydrogenase into the host gene encoding a lactate dehydrogenase to thereby introduce NAD-linked formate dehydrogenase activity and knock out lactate dehydrogenase activity in a single cloning step. Preferably, the gene encoding an NAD-linked formate dehydrogenase is flanked upstream by at least the promoter region of the gene encoding a lactate dehydrogenase, so that following integration by recombination the gene encoding an NAD-linked formate dehydrogenase is operably linked to the promoter. In a particularly preferred embodiment, the downstream portion of the lactate dehydrogenase gene is one obtainable by amplification of the ldh gene using primers comprising, consisting essentially of or consisting of the nucleotide sequence set forth as SEQ ID NO: 8 and 9, using the strain LLD-R as template. The upstream flanking region, which preferably incorporates the ldh promoter, preferably comprises at least 100, 200, 300, 400, 500, 600, 700 or 750 nucleotides of the appropriate ldh upstream regions to maximise efficiency of integration by recombination with the host genome. This upstream region may be dependent upon the sequence context of the ldh gene in the specific thermophilic microorganism of interest, as would be readily determined by a skilled person. Thus, the skilled person with knowledge of the ldh gene sequence would readily determine appropriate flanking regions to allow integration by recombination. For example, published genomic sequences may be studied, sequencing reactions carried out or flanking regions amplified by PCR using primers derived from the ldh gene sequence. Thus the fdh gene becomes interposed between two nucleotide sequences derived from the ldh gene such that the fdh gene replaces, in frame, at least part of the ldh gene.

The DNA construct thus generally comprises a gene integration cassette in which the gene of interest (gene encoding an NAD-linked formate dehydrogenase) is inserted within the coding sequence (ORF) of a gene to be knocked out during integration (lactate dehydrogenase gene in this instance). Upon integration through recombination, expression of the gene of interest is in effect under the control of the knocked out gene. Such a construct may be of general applicability in the circumstance where one gene needs to be knocked out in favour of expression of a heterologous gene. In one preferred embodiment, the gene encoding an NAD-linked formate dehydrogenase encodes a thermostable NAD-linked formate dehydrogenase. The discussion of the thermostable NAD-linked formate dehydrogenases provided herein thus applies mutatis mutandis to this aspect of the invention. In particular, in one embodiment, the gene encoding a thermostable NAD-linked formate dehydrogenase comprises, consists essentially of or consists of the nucleotide sequence set forth as SEQ ID NO:1 or 2. A particularly preferred DNA construct is plasmid pUCK-LF1 (as described in the experimental section below and in FIG. 8).

For all DNA constructs of the invention a preferred form is an expression vector. Thus, the DNA constructs allow reliable expression of the gene encoding a thermostable NAD-linked formate dehydrogenase in a microorganism transformed with the construct. In a particularly preferred embodiment, the DNA construct is a plasmid. Preferably, the DNA construct can only replicate in the host thermophilic microorganism through recombination with the genome of the host thermophilic microorganism.

The DNA constructs of the invention also preferably incorporate a suitable reporter gene as an indicator of successful transformation. In one embodiment, the reporter gene is an antibiotic resistance gene, such as a kanamycin or ampicillin resistance gene. Other reporters, such as green fluorescent protein (GFP) and beta-galactosidase (lacZ) may be utilised as appropriate. The DNA constructs may incorporate multiple reporter genes, as appropriate. Loss of reporter function is, in subsequent generations, indicative of integration of the gene encoding a thermostable NAD-linked formate dehydrogenase, together with appropriate flanking regions.

In a still further aspect, the invention relates to a microorganism comprising a DNA construct of the invention. Preferred recipient microorganisms are heteroloactate fermentative microorganism. In particular, the invention preferably relates to thermophilic bacteria, such as those of the genus Bacillus and especially Bacillus stearothermophilus. The bacterium may be derived from strain LLD-R or LLD-15 for example. In a further embodiment, the thermophilic microorganism is Geobacillus thermoglucosidasius.

In a yet further aspect, the invention relates to the use of a microorganism of the invention or a thermophilic microorganism of the invention in fermentation, and in particular for the production of ethanol.

Similarly, the invention relates to a fermentation process for the production of ethanol comprising supplying a thermophilic microorganism of the invention or a microorganism of the invention with sugars. Microorganisms constructed according to the present invention are particularly suitable for high ethanol yield and volumetric productivity under optimal growth conditions. Accordingly, any microorganism of the invention may be used in any fermenter configuration, such as batch, fed-batch or continuous fermentation processes. In one preferred embodiment, the fermentation process is a fed-batch process.

One of the principal benefits of using microorganisms such as thermophilic bacteria to produce bioethanol is that, unlike yeasts, they are capable of fermenting a wide range of sugars derived from agricultural waste products such as hemicelluloses. Accordingly, in one embodiment the sugars used in the fermentation processes of the invention are derived from biomass. In a further embodiment, fermentation is of mixed sugars. In a specific embodiment, the mixed sugars include pentose sugars, preferably a majority of pentose sugars.

In a further embodiment, the fermentation process is maintained in redox balance. This is particularly critical with thermophiles since, unlike mesophiles, sugar uptake appears to be unregulated in these microorganisms. Preferably, this is achieved through use of feedback sensors.

Whilst thermophilic bacteria have low tolerance to ethanol, this can conveniently be overcome in the fermentation processes of the invention by regular or continuous removal of ethanol. This ensures that the ethanol concentration in the fermentation is kept below the ethanol tolerance of the thermophilic microorganism or microorganism of the invention. Ethanol may be continuously and conveniently removed from the high temperature fermentation by evaporation or distillation, such as membrane and/or mild vacuum evaporation for example.

The invention will now be described with reference to the following non-limiting description and figures.

### BRIEF DESCRIPTION OF THE FIGURES

FIG. 1 shows the effect of various conditions on metabolic pathways in a thermophilic microorganism in which the lactate dehydrogenase pathway has been inactivated. The shade and thickness of the arrows indicate the relative dominance of the respective metabolic pathways.

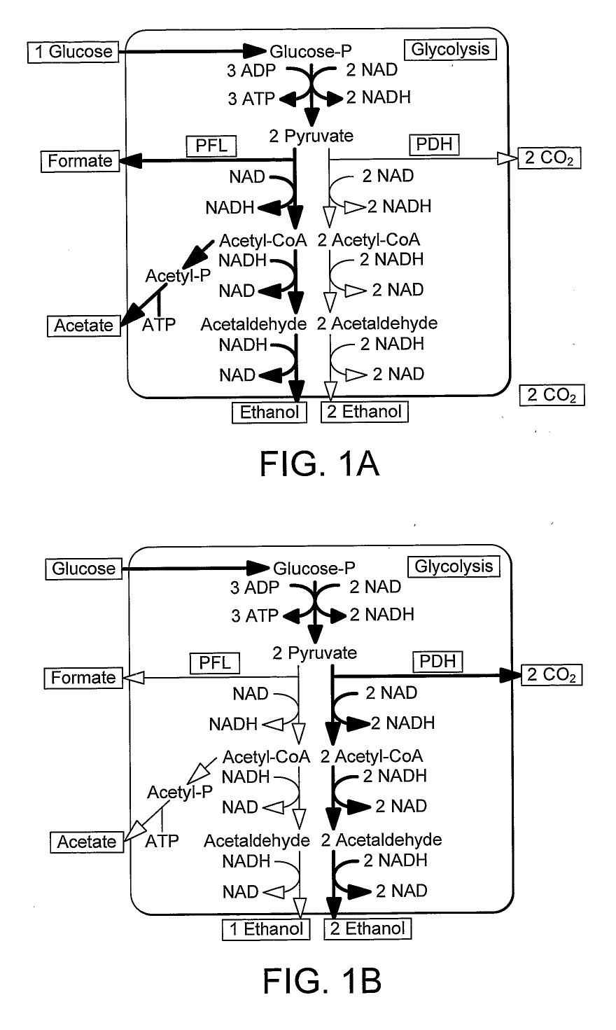

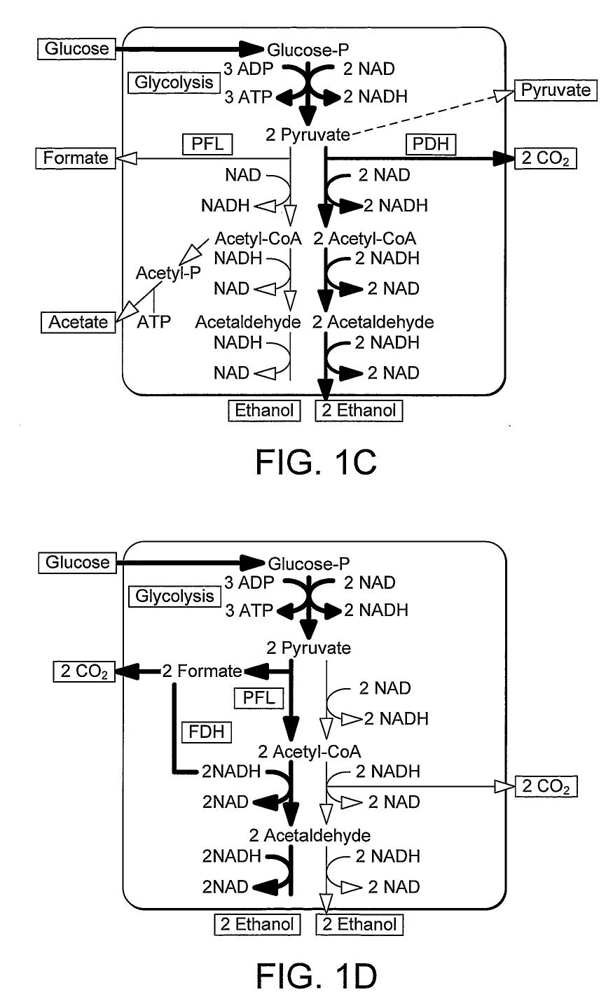

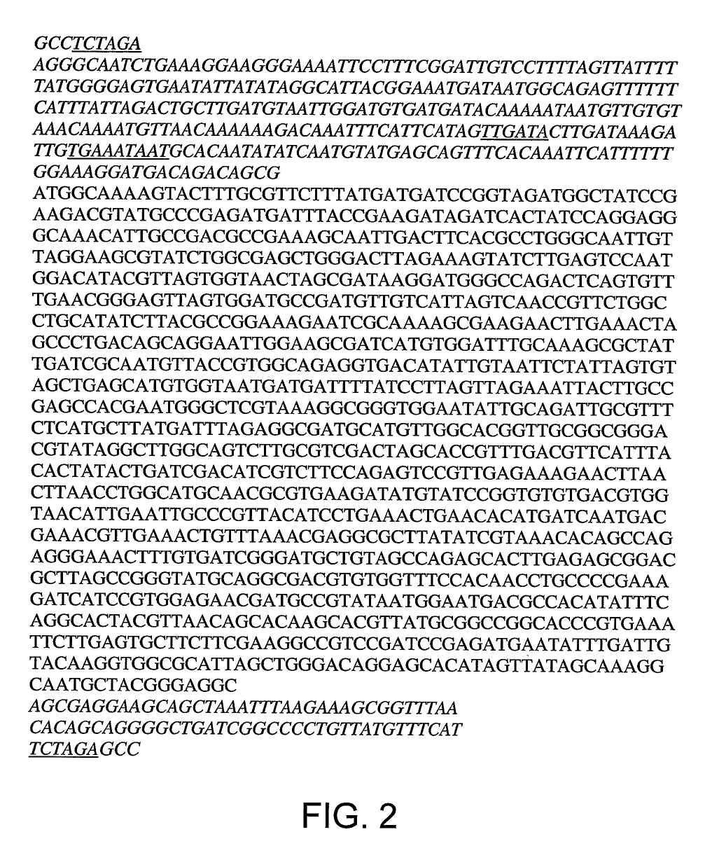

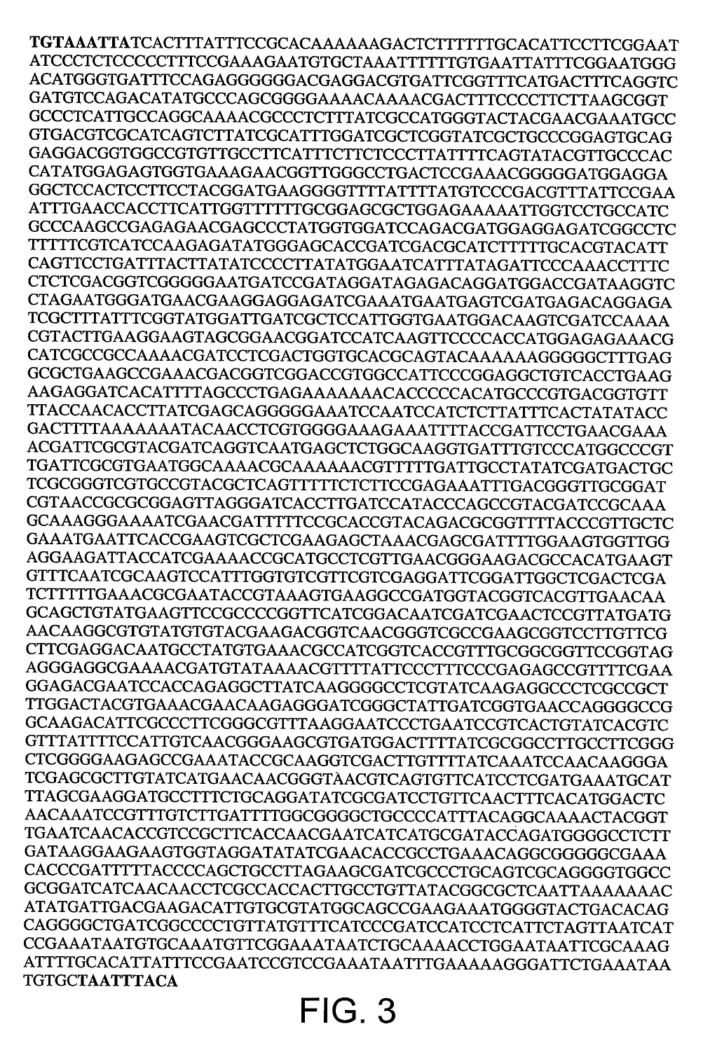

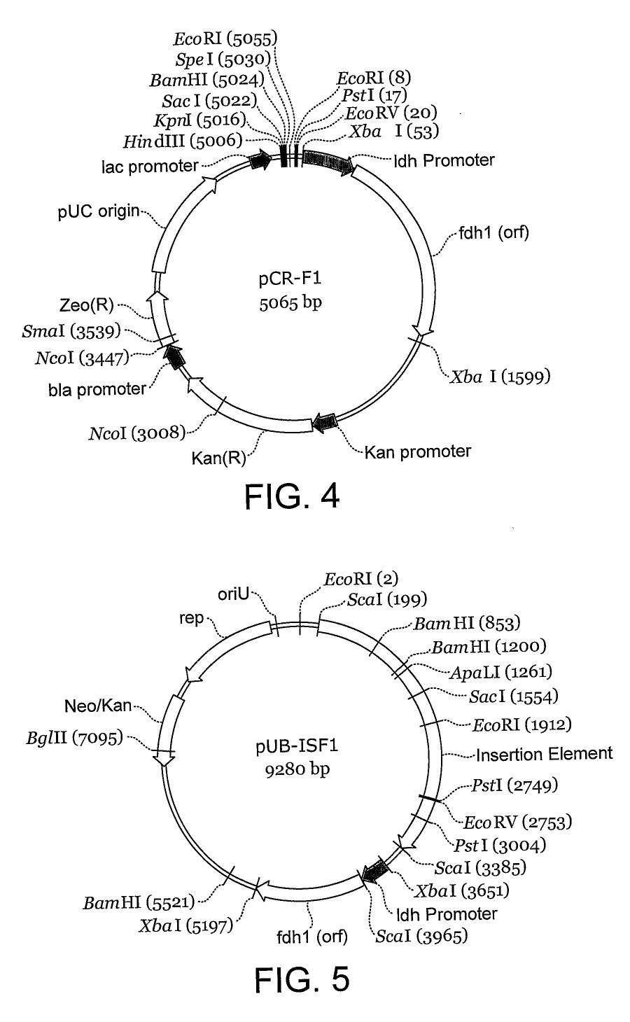

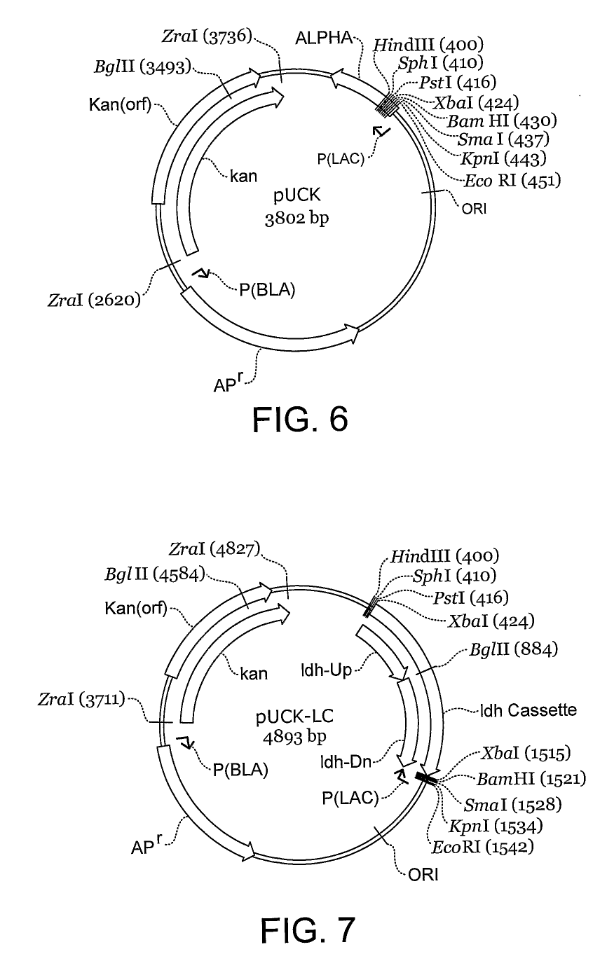

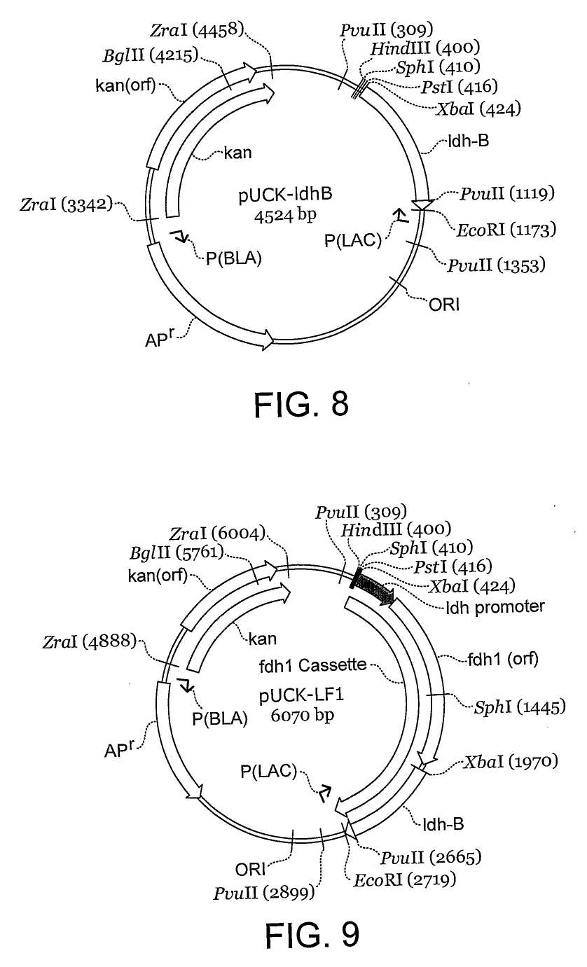

A. Metabolic pathways active at neutral pH and in the presence of low sugars. Here the pyruvate formate lyase pathway dominates.
B. Metabolic pathways active at low pH and in the presence of low sugars. Here an anaerobic pyruvate dehydrogenase pathway dominates.
C. Metabolic pathways active at low pH and in the presence of high sugars. Here the cells experience metabolic stress and fall out of redox balance leading to so called “redox death”.
D. Metabolic pathways active in thermophilic microorganisms of the present invention, at neutral pH and in the presence of high sugars. Here, the novel PFL-FDH pathway is dominant and ethanol and CO₂ are the only anaerobic products.

FIG. 2 sets forth the nucleotide sequence of the synthetic fdh gene produced by codon optimisation to maximise thermostability. The DNA sequence of the fdh open reading frame is flanked by promoter and terminator (italics) regions. −35 and −10 boxes of the promoter are underlined. To clone the construct in a suitable vector, Xba1 sites were introduced on both sides of the sequence.

FIG. 3 shows the nucleotide sequence of the Insertion Sequence (IS) of B. stearothermophilus Strain LLD-15. The 9 bp inverted repeat ends are shown in bold font.

FIG. 4 is a schematic representation of the pCR-Blunt derivative plasmid pCR-F1. The plasmid includes a codon optimised fdh1 gene under the control of the ldh promoter, cloned into pCR-Blunt at the unique Xba1 site.

FIG. 5 is a schematic representation of the pUB110 derivative plasmid pUB-ISF1. The plasmid includes a codon optimised fdh1 gene under the control of the ldh promoter, derived from the pCR-F1 plasmid and also an insertion sequence (IS) derived from the known Bacillus strain LLD-15.

FIG. 6 is a schematic representation of the pUC18 derivative plasmid called pUCK. The plasmid includes a kanamycin resistance gene cloned from plasmid pUB110 into the unique Zra1 restriction sitein pUC18.

FIG. 7 is a schematic representation of the pUCK derivative pUCK-LC. The plasmid carries an ldh gene with a deletion of 363 bp in the middle of the ORF.

FIG. 8 is a schematic representation of the pUCK derivative pUCK-ldhB. The plasmid contains 750 bp of the ldh gene, including the downstream region of the gene.

FIG. 9 is a schematic representation of the pUCK-LF1 plasmid. This plasmid is a pUCK derivative incorporating a gene integrating cassette containing the fdh gene under the control of the ldh promoter.

### DESCRIPTION OF THE INVENTION

### Materials

Media and buffers

LB medium: Tryptone 10 g; Yeast Extract 5 g; NaCl 10 g; deionised water to 1 L

Adjusted pH to 7 and autoclaved to sterilize

For plate medium 20 g/l agar was added to the medium before autoclaving, cooled to 55° C. and poured into sterile Petri dishes (approx. 20 ml/plate).

For LB-amp plates filter-sterilised ampicillin solution was added to final concentration of 50 μg/ml before pouring the Petri plates.

SOC Medium: Tryptone 2.0 g; Yeast Extract 0.5 g; NaCl 0.05 g; MgCl₂.6H₂O 0.204 g;

MgSO₄.7H₂O 0.247 g; Glucose 0.36 g; deionised H₂O to 100 ml. Dissolved, adjusted the pH to 7.0 and filter sterilised.

TGP Medium: Tryptone 17 g; Soya peptone 3 g; K₂HPO₄ 2.5 g; NaCl 5 g; Na pyruvate 4 g; glycerol 4 ml; deionised water to 1 L. Adjusted pH to 7 and autoclaved to sterilize.

For plate medium, 20 g/l agar was added in the medium before autoclaving, cooled to 55° C. and poured into sterile Petri dishes (approx. 25 ml/plate).

For TGP-kan plates, filter-sterilised kanamycin solution to final concentration of 10 μg/ml was added before pouring the Petri plates.

TH buffer: Trehalose 272 mM; HEPES (pH 7.5 with KOH) 8 mM; double distilled H₂O to 1 L

### Microbial Strains

E. coli DH5-alpha-Chemically competent cells were purchased from Invitrogen (Cat. 18265-017).

Bacillus subtilis subsp. Subtilis-German culture collection, DSMZ (DSM No. 10)

Bacillus stearothermophilus. strain LLD-R—Deposited as NCIMB 12403

Bacillus stearothermophilus. strain LLD-15—Deposited as NCIMB 12428

### Plasmids

Plasmid pCR-Blunt and pCR-TOPO2 were obtained from Invitrogen

Plasmid pUB110—Bacillus subtilis BD170 strain harbouring this plasmid was obtained from the German culture collection, DSMZ (DSM No. 4514).

Plasmid pUC18 was obtained from Sigma-Aldrich.

### Example 1

### Construction of a Synthetic Formate Dehydrogenase Gene (FIG. 2)

An amino acid sequence (NCBI Protein Database Accession No P33160—SEQ ID NO:3) of Pseudomonas sp 101 formate dehydrogenases was back translated into DNA sequence with optimised codons for Geobacillus thermoglucosidasius. A promoter and a rho-independent terminator region from a Bacillus strain were added, upstream and downstream of the translated sequence respectively (FIG. 2). The novel sequence showed less than 40% similarity with known fdh gene sequences (37% identity with known fdh1 gene). Xba1 sites were designed into both sides of the construct to facilitate its cloning into suitable vectors.

The desired sequence was synthesized using the method of Gao et al (see Xinxin Gao, Peggy Yo, Andrew Keith, Timothy J. Ragan and Thomas K. Harris (2003). Nucleic Acids Research, 31 (22), e143) and cloned into pCR-Blunt at its unique Xba1 position. The resulting vector pCR-F1 (FIG. 4) was introduced into E. coli DH5 alpha cells and the positive clones were confirmed by PCR and restriction analysis.

Two alternative strategies are available to insert and express this synthetic fdh gene in the genome of target Bacilli as shown in the following examples.

### Example 2

### Insertion of the fdh Gene into Multiple (IS) Sites

This strategy applies to strains such as Bacillus stearothermophilus strain LLD-R that contain an Insertion Sequence (IS) that frequently recombines at multiple insertion sites. A vector carrying the fdh gene and this IS sequence is expected to integrate stably at one or more of such locations

Construction of Plasmid pUB-ISF1 (FIG. 5)

Firstly, the known Insertion Sequence of strain LLD-R (SEQ ID NO: 5 and FIG. 3) is PCR amplified using a forward primer (AGTACTGAAATCCGGATTTGATGGCG—SEQ ID NO:6) and a reverse primer (AGTACTGCTAAATTTCCAAGTAGC—SEQ ID NO:7) with B. stearothermophilus strain LLD-15 as the template. Sca1 restriction sites are introduced in the both ends of the sequence. The PCR product is first cloned in plasmid pCR-TOPO2.1 and the resulting plasmid pCR-IS is then introduced into E. coli DH5 alpha cells and used to isolate the IS region by Sca1 restriction digestion. The isolated IS is then cloned in pUB110 at its unique Sca1 site and the resulting plasmid pUB-IS is introduced into Bacillus subtilis.

Then a 1.5 kb fragment containing the ldh promoter and the fdh gene are digested from the pCR-F1 plasmid using Xba1 restriction enzyme, and cloned in plasmid pUB-IS that was already linearised with the same enzyme. The resulting plasmid pUB-ISF1 (FIG. 5) is then introduced into B. subtilis and positive clones are selected on TGP-kan plates and confirmed by PCR and restriction analysis.

Integration of the fdh Gene into Strain LLD-R

Plasmid pUB-ISF1 is then methylated in vitro with HaeIII methylase enzyme and then Bacillus stearothermophilus strain LLD-R or its ldh-deleted strains (see Example 3) cells are transformed with the methylated pUB-ISF1 plasmid. Positive clones are selected after 48 hours on TGP-Kan plates at 50° C., and analysed by PCR amplification of the fdh gene.

The fdh gene is then integrated in the chromosome by growing a transformed clone in TGP-Kan medium at 60-65° C. for a few generations and selecting on TGP-Kan plates. The positive clones are analysed for presence of the Edh gene and then screened for ethanol production and C5 (pentose) and C6 (hexose) sugar utilisation in shake flasks and in fermenters.

### Example 3

### Construction of ldh-Deleted Strains

The first step is to clone a Bacillus kanomycin resistance marker (kan) and a cassette carrying the ldh gene of B. stearothermophilus strain LLD-R into plasmid pUC18, which can replicate only in gram negative microorganisms.

Construction of a Bacillus Cloning Vector. Plasmid pUCK (FIG. 6).

A kanamycin resistance gene (kan) was cloned in plasmid pUC18 at its unique Zra1 site which is outside of any coding region and of the reporter gene (lacZ) in the plasmid. To clone the kan gene, a 1.13 kb fragment containing the kanamycin resistance gene was PCR amplified with the primers:

kan-BsZ-F
(ACACAGACGTCGGCGATTTGATTCATAC-SEQ ID NO:10)
and
kan-BsZ-R
(CGCCATGACGTCCATGATAATTACTAATACTAGG-SEQ ID NO:11)

using plasmid pUB110 as template. The Zra1 sites were introduced at both ends of the kan gene through the primers. The PCR product was then digested with Zra1 restriction endonuclease enzyme and ligated with previously Zra1-digested and dephosphorylated plasmid pUC18. The resulting plasmid pUCK (FIG. 6) was then introduced into E. coli DH5 alpha cells. Positive clones were selected on LB-amp plates and confirmed by PCR and restriction analysis.
Construction of Plasmid pUCK-LC (FIG. 7) Which Carries a Deleted ldh Gene

A 1.36 kb ldh cassette was designed to contain the whole ldh gene of strain LLD-R from which 363 bp of its ORF was deleted plus its flanks. The cassette was constructed by PCR amplification of the upper and lower regions of the ldh gene using strain LLD-R as template. These regions were then ligated and cloned in plasmid pUCK. BglII sites were introduced into the inner primers. The upper region was PCR amplified using the following primers:

LC-U-F1
(AGGGCAATCTGAAAGGAAGGGAAAATTCC-SEQ ID NO:12)
and
LC-UB-R1
TGCACAGATCTCCACCAAATCGGCGTC-SEQ ID NO:13).

The lower region was PCR amplified using the following primers:

LC-DB-F1
(TTGAGCAGATCTTGATGCAAAACGATAAC-SEQ ID NO:14)
and
LC-D-R1
(TAAAGCCGATGAGCAGCAGTTGAAG-SEQ ID NO:15).

The PCR products were digested with BglII restriction endonuclease enzyme and ligated using T4 DNA ligase enzyme. Using the ligate as template, the ldh-cassette was then PCR amplified using as primers:

LC-UX-F2
(ATATTATCTAGACATTACGGAAATGATAATGGC-SEQ ID NO:16)
and
LC-DX-R2
(TCACAATCTAGACAATCGGCCATAAAC-SEQ ID NO:17).

XbaI sites were introduced at the both ends of the cassette via the primers. The PCR product was then digested with XbaI enzyme and cloned into plasmid pUCK pre-digested with the same enzyme and dephosphorylated. The resulting plasmid pUCK-LC was then introduced into E. coli DH5 alpha. The positive clones were selected on LB-amp plates and confirmed by PCR and restriction analysis.

### Construction of ldh Deleted Strains

Plasmid PUCK-LC is methylated in vitro with HaeIII methylase enzyme and wild type thermophile cells (e.g. strain LLD-R) are transformed into the methylated plasmid by electroporation (1000 V, 201 ohms, 25 micro-Faraday, and 5 milli-seconds). Positive clones are selected on TGP-Kan plates at 65° C. and confirmed as single cross-over events by PCR amplification of the kan gene.

To achieve gene deletion by double cross-over, the positive clones are grown in TGP medium for a few generations (about 5 sub-cultures) and clones which can grow on TGP plates but not on TGP-kan plates are selected. The positive clones are then confirmed as ldh gene deletions and for the absence of the kanamycin gene by PCR analysis. The clones are then characterised for ethanol production and C5 and C6 sugar utilisation in shake flasks and in fermenters.

### Example 4

### Simultaneous Insertion of the fdh Gene and Deletion of the ldh Gene

This alternative strategy is broadly applicable to a wide class of heterolactate fermentative microorganisms as well as thermophilic Bacilli, though the latter will be used as illustration.

Construction of Plasmid pUCK-LF (FIG. 9)

A gene integrating cassette containing the fdh gene plus the whole ldh gene and about 300 bp of upstream and downstream flanking regions is cloned into plasmid pUCK. In this construct, the first 450 bp of the ldh open reading frame are replaced with the fdh gene in such a way that the gene expression becomes under control of the ldh gene promoter.

To achieve this a DNA fragment of about 750 bp containing the downstream region of the ldh gene is PCR amplified using;

LDHB-X-F1 (GAACGATTCTAGATACAGCAAGATTCCGC—SEQ ID NO:8) and LDHB-E-R1 (GTTTGCGAATTCATAGACGGACGCAG—SEQ ID NO:9) as primers and Bacillus stearothermophilus strain LLD-R as template. Xba1 and EcoR1 sites are thus introduced in the ends of the PCR fragment. The PCR fragment is then digested and directionally cloned in plasmid pUCK between the Xba1 and EcoR1 sites. The resulting plasmid, pUCK-ldhB (FIG. 8) is introduced into E. coli DH5 alpha and positive clones are selected on LB-amp plates and confirmed by PCR and restriction

Then, a 1.5 kb fragment containing the ldh promoter and the fdh gene are digested out from the pCR-F1 plasmid using Xba1 restriction enzyme and cloned into plasmid pUCK-ldhB (FIG. 8) which was already linearised with the same enzyme. The resulting plasmid pUCK-LF1 (FIG. 9) is introduced into E. coli DH5 alpha cells and clones are selected on LB-Amp plates. Positive clones and the correct orientation of the construct are confirmed by PCR and restriction analysis.

Construction of Strains that Make Ethanol by the Novel PFL-FDH Pathway.

Plasmid pUCK-LF1 is methylated in vitro with HaeIII methylase enzyme, and target wild type cells (e.g. strain LLD-R cells) are transformed with the methylated plasmid and selected on TGP-Kan plates at 60° C. The positive clones represent single cross-over events and are analysed by PCR amplification of the fdh gene.

To achieve double cross-over gene integration, clones that grow on TGP plates but not on TGP-Kan plates are selected. The positive clones are then confirmed for the presence of the fdh gene and absence of the kanamycin gene. Finally the clones are screened for ethanol production and C5 and C6 sugar utilisation in shake flasks and in fermenters.

All references are incorporated herein in their entirety.

1. A thermophilic microorganism lacking lactate dehydrogenase activity characterised in that the thermophilic microorganism contains a gene encoding an NAD-linked formate dehydrogenase.

2. The thermophilic microorganism of claim 1 which has pyruvate formate lyase activity.

3. The thermophilic microorganism of claim 1 wherein the gene encoding an NAD-linked formate dehydrogenase is integrated into the genome of the thermophilic microorganism.

4. The thermophilic microorganism of claim 1 wherein the gene encoding an NAD-linked formate dehydrogenase is expressed from its own promoter or from a promoter of the thermophilic microorganism.

5. The thermophilic microorganism of claim 1 wherein the gene encoding an NAD-linked formate dehydrogenase is inserted into the lactate dehydrogenase gene of the thermophilic microorganism, thus inactivating the lactate dehydrogenase activity of the thermophilic microorganism.

6.-35. (canceled)

36. The thermophilic microorganism of claim 1 wherein the gene encoding an NAD-linked formate dehydrogenase comprises the nucleotide sequence set forth as SEQ ID NO: 1 or 2.

37. The thermophilic microorganism of claim 1 which has been transformed with a DNA construct comprising a gene encoding an NAD-linked formate dehydrogenase operably linked to an upstream region of a gene encoding a lactate dehydrogenase wherein the upstream region includes the promoter and further comprising at least part of the lactate dehydrogenase gene downstream of the gene encoding an NAD-linked formate dehydrogenase such that the gene encoding an NAD-linked formate dehydrogenase is interposed between a sufficient portion of the lactate dehydrogenase gene on either side to facilitate integration of the gene encoding an NAD-linked formate dehydrogenase by recombination with a lactate dehydrogenase gene in the genome of the thermophilic microorganism.

38. The thermophilic microorganism of claim 1 which is a thermophilic bacterium of the genus Bacillus.

39. A gene encoding a thermostable NAD-linked formate dehydrogenase comprising the nucleotide sequence set forth as SEQ ID NO:1.

40. A DNA construct comprising a regulatory sequence operably linked to a gene encoding a thermostable NAD-linked formate dehydrogenase comprising the nucleotide sequence set forth as SEQ ID NO:1.

41. A DNA construct comprising a gene encoding an NAD-linked formate dehydrogenase, optionally a thermostable NAD-linked formate dehydrogenase, and an insertion sequence, wherein the insertion sequence facilitates integration of the gene encoding an NAD-linked formate dehydrogenase into the genome of a thermophilic microorganism transformed with the DNA construct.

42. A DNA construct comprising a gene encoding an NAD-linked formate dehydrogenase, optionally a thermostable NAD-linked formate dehydrogenase, operably linked to an upstream region of a gene encoding a lactate dehydrogenase, wherein the upstream region includes the promoter.

43. A microorganism comprising the DNA construct as defined in claim 40.

44. A microorganism comprising the DNA construct as defined in claim 41.

45. A microorganism comprising the DNA construct as defined in claim 42.

46. A fermentation process for production of ethanol comprising supplying a thermophilic microorganism as claimed in claim 1 with sugars.

47. A fermentation process for production of ethanol comprising supplying a thermophilic microorganism as claimed in claim 43 with sugars.

48. A fermentation process for production of ethanol comprising supplying a thermophilic microorganism as claimed in claim 44 with sugars.

49. A fermentation process for production of ethanol comprising supplying a thermophilic microorganism as claimed in claim 45 with sugars.
